(19) United States
(12) Patent Application Publication
(10) Pub. No.: US 2009/0192185 A1
(43) Pub. Date: Jul. 30, 2009

(54) MEDICAL PRODUCT

(75) Inventors: Thomas NILSSON, Meilen (CH); Mattias MYRMAN, Tyresoe (SE); Sven CALANDER, Straengnaes (SE); Alf NIEMI, Straengnaes (SE); Bernd ZIERENBERG, Bingen am Rhein (DE); Holger MEMMESHEIMER, Ockenheim (DE); Christine LUNKENHEIMER, Ingelheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Boehringer Ingelheim International GmbH, Ingelheim am Rhein (DE)

(21) Appl. No.: 12/417,814

(22) Filed: Apr. 3, 2009

Related U.S. Application Data
(63) Continuation of application No. 12/255,086, filed on Oct. 21, 2008.
Application No. 12/255,086 is a continuation of application No. 10/728,986, filed on Dec. 8, 2003, now abandoned.

(30) Foreign Application Priority Data
Dec. 3, 2003 (SE) 0303269-5

(51) Int. Cl. A61K 31/46; A61P 11/00

(52) U.S. Cl. 514/291

(57) ABSTRACT

The invention discloses a medical product for use in a treatment of respiratory disorders, and comprises a metered dose of a tiotropium dry powder formulation, directly loaded and sealed into a container made to act as a dry high barrier seal to prevent the capture and ingress of moisture into the tiotropium powder. The dose of tiotropium is further adapted for inhalation and the container is so tight that the efficacy of the dose when delivered is unaffected by moisture. In a further aspect of the invention a type of inhaler is illustrated, which may accept at least one sealed, moisture-tight container of a dose of tiotropium, to deliver the dose with a consistent fine particle dose, over the expected shelf life of the product.

### REFERENCE TO PRIOR APPLICATIONS

This application is a Continuation application of U.S. application Ser. No. 12/255,086, filed Oct. 21, 2008, now pending; which is a Continuation application of U.S. application Ser. No. 10/728,986, filed Dec. 8, 2003, now abandoned; which claims the benefit of SE 0303269-5 filed Dec. 3, 2003, both of which are incorporated herein by reference.

### TECHNICAL FIELD

The present invention relates to a medical product comprising inhalable doses of tiotropium loaded in a moisture-tight, dry container and in particular, a metered dry powder medicinal dose of tiotropium bromide being adapted for administration by a dry powder inhaler device.

### BACKGROUND

Asthma and chronic obstructive pulmonary disease (COPD) affect more than 30 million people in the United States. More than 100,000 deaths each year are attributable to these conditions. Obstruction to airflow through the lungs is the characteristic feature in each of these airway diseases, and the medications utilized in treatment are often similar.

Chronic obstructive pulmonary disease (COPD) is a widespread chronic lung disorder encompassing chronic bronchitis and emphysema. The causes of COPD are not fully understood. Experience shows that the most important cause of chronic bronchitis and emphysema is cigarette smoking. Air pollution and occupational exposures may also play a role, especially when combined with cigarette smoking. Heredity also causes some emphysema cases, due to alpha1 anti-trypsin deficiency.

Administration of asthma drugs by an oral inhalation route is very much in focus today, because of advantages offered like rapid and predictable onset of action, cost effectiveness and high level of comfort for the user. Dry powder inhalers (DPI) are especially interesting as an administration tool, compared to other inhalers, because of the flexibility they offer in terms of nominal dose range, i.e. the amount of active substance that can be administered in a single inhalation.

Anticholinergic agents, e.g. tiotropium, especially tiotropium bromide, are effective bronchodilators. These medicaments have a relatively fast onset and long duration of action, especially tiotropium bromide, which may be active for up to 24 hours. Anticholinergic agents reduce vagal cholinergic tone of the smooth muscle, which is the main reversible component of COPD. Anticholinergic agents have been shown to cause quite insignificant side effects in clinical testing, dryness of mouth and constipation are perhaps the most common symptoms. Because it is often very difficult to diagnose asthma and COPD correctly and since both disorders may co-exist, it is advantageous to treat patients suffering temporary or continuous bronchial obstruction resulting in dyspnoea with a small but efficient dose of a long-acting anticholinergic agent, preferably tiotropium bromide, because of the small adverse side effects.

Tiotropium bromide is the preferred anticholinergic agent because of its high potency and long duration. However, tiotropium is difficult to formulate in dry powder form to provide acceptable performance in terms of dose efficacy using prior art DPIs. Dose efficacy depends to a great deal on delivering a stable and high fine particle dose (FPD) out of the dry powder inhaler. The FPD is the respirable dose mass out of the dry powder inhaler with an aerodynamic particle size below 5 μm. Thus, when inhaling a dose of dry medication powder it is important to obtain by mass a high fine particle fraction (FPF) of particles with an aerodynamic size preferably less than 5 μm in the inspiration air. The majority of larger particles (>5 μm) does not follow the stream of air into the many bifurcations of the airways, but get stuck in the throat and upper airways, where the medicament is not giving its intended effect, but may instead be harmful to the user. It is also important to keep the dosage to the user as exact as possible and to maintain a stable efficacy over time, and that the medicament dose does not deteriorate during normal storage. For instance, Boehringer Ingelheim KG (BI) markets tiotropium bromide under the proprietary name of SPIRIVA®. Surprisingly, in a recent investigation into the inhalability of SPIRIVA® we have found that the SPIRIVA®/HANDIHALER® system from BI for administration by inhalation of doses contained in gelatin capsules shows poor performance and has short in-use stability.

Thus, there is a need for improvement regarding a medical product comprising inhalable dry powder doses of tiotropium bromide, for instance SPIRIVA®, and suitably adapted inhaler devices for the purpose of administration.

### SUMMARY

The present invention discloses a medical product for use in the treatment of respiratory disorders, and comprises a metered dose of a tiotropium dry powder formulation, directly loaded and sealed into a moisture-tight, dry container acting as a dry, high barrier seal against moisture. The container itself does not emit water, which may affect the tiotropium powder inside. Thus, the container does not release any water to the dose and ingress of moisture from the exterior into the container is thereby prevented.

The dose of tiotropium is further intended for inhalation and the container is so dry and tight that the efficacy of the dose when delivered is unaffected by moisture.

In another aspect of the invention a type of inhaler is disclosed, which may accept at least one sealed, moisture-tight, dry container of a dose of tiotropium, e.g. SPIRIVA®, and deliver said dose with a consistent FPD, over the expected shelf life of the product.

In a further aspect of the invention tiotropium may be mixed or formulated with at least one additional pharmacologically active ingredient(s) with an object of combining tiotropium with other medicament(s) to be used in the treatment of respiratory disorders. The present invention encompasses such use of tiotropium in a combination of medicaments directly loaded into a sealed, moisture-tight, dry container for insertion into a DPI, the combination adapted for inhalation by the user.

The present medical product is set forth by the independent claims 1 and 2 and the dependent claims 3 to 13, and a pharmaceutical combination is set forth by the independent claims 14 and 15 and the dependent claims 16 to 25.

### BRIEF DESCRIPTION OF THE DRAWINGS

The invention, together with further objects and advantages thereof, may best be understood by referring to the following detailed description taken together with the accompanying drawings, in which:

FIG. 1 illustrates in a graph the results of tests S1 to S5 and HBS1 to HBS3;

FIG. 2 illustrates in top and side views a first embodiment of a dose deposited onto a dose bed and a high barrier seal; and

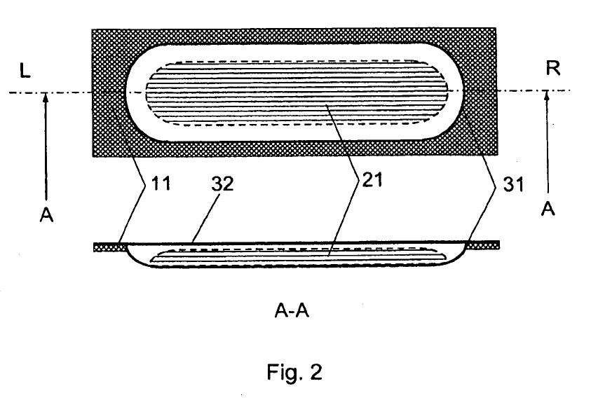

FIG. 3 illustrates in top and side views a second embodiment of a dose onto a dose bed and a high barrier seal.

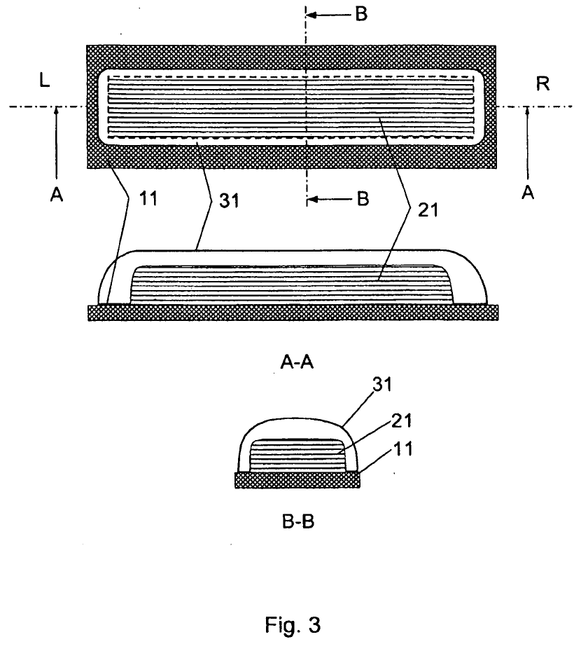

### DETAILED DESCRIPTION

Tiotropium is a new important anticholinergic substance for treatment of asthma and COPD but tiotropium is known in the industry to have problems maintaining in-use stability due to sensitivity to moisture. This fact is also documented in the report ‘COLLEGE TER BEOORDELING VAN GENEESMIDDELEN MEDICINES EVALUATION BOARD; PUBLIC ASSESSMENT REPORT; SPIRIVA 18 μg, inhalation powder in hard capsules; RVG 26191’ (2002-05-21) on page 6/28 under ‘Product development and finished product’ a very short in-use stability of the SPIRIVA® product (9 days) is reported and a brittleness of the capsule in the blister pack and a very low FPD:‘about 3 ug’.

Details about an inhalation kit comprising inhalable powder of tiotropium and use of an inhaler for the administration of tiotropium may also be studied in the international publication WO 03/084502 A1. Details about tiotropium compounds, medicaments based on such compounds, the use of compounds and processes for preparing compounds may be studied in the European Patent Application 0 418 716 B1.

In the light of the above information given in the quoted report a test program was set up for the physical stability of the SPIRIVA® product with respect to the compatibility of the formulation together with the components of the device according to Food and Drug Administration (FDA) ‘Guidance for Industry; Metered Dose Inhaler (MDI) and Dry Powder Inhaler (DPI) Drug Products; Chemistry, Manufacturing, and Controls Documentation’ page 37/62 ‘Drug product stability’ lines 1209-1355. In ‘Guidance for Industry; Stability Testing of Drug Substances and Drug Products; DRAFT GUIDANCE; B. Container/Closure’ pages 35 and 36/110 lines 1127-1187, FDA states:‘Stability data should be developed for the drug product in each type of immediate container and closure proposed for marketing, promotion, or bulk storage. The possibility of interaction between the drug and the container and closure and the potential introduction of extractables into the drug product formulations during storage should be assessed during container/closure qualification studies using sensitive and quantitative procedures.’ and further ‘Loss of the active drug substance or critical excipients of the drug product by interaction with the container/closure components or components of the drug delivery device is generally evaluated as part of the stability protocol. This is usually accomplished by assaying those critical drug product components, as well as monitoring various critical parameters (e.g., pH, preservative, effectiveness). Excessive loss of a component or change in a parameter will result in the failure of the drug product to meet applicable specifications.’

According to FDA publication ‘Guidance for Industry; Stability Testing of Drug Substances and Drug Products’ a 3 week test program in accelerated conditions (40±2°/75±5 RH) for the container closure of the SPIRIVA® product in this case the capsule and the blister pack and the impact of the capsule and the blister package on the FPD was set up and tested.

### Execution of Tests

SPIRIVA® powder formulation in bulk and SPIRIVA® capsules from our local pharmacy where introduced to the laboratory together with the HANDIHALER®. The laboratory was set up to perform in-vitro tests according to European Pharmacopoeia (EP) and US Pharmacopoeia (USP) using two Andersen cascade impactors. All analytical work where then performed according to standardized methods for Physical Tests and Determinations for Aerosols, metered-dose inhalers and dry powder inhalers described in pharmacopoeias (e.g. USP 2002<601>) using a state of the art High Performance Liquid Chromatograph (HPLC) system.

### SPIRIVA® Tests

Test S1
Aerodynamic fine particle fraction of metered and delivered dose out of HANDIHALER® using SPIRIVA® formulation from bulk powder loaded into originator capsules during relative humidity below 10%. The test was performed with 4 kPa pressure drop over the HANDIHALER® at room temperature and laboratory ambient conditions.

Test S2
Aerodynamic fine particle fraction of metered and delivered dose out of HANDIHALER® using commercial SPIRIVA® capsules purchased from our local pharmacy. Test performed with 4 kPa pressure drop over the HANDIHALER® at room temperature and laboratory ambient conditions.

Test S3
An in-use stability test of the aerodynamic fine particle fraction of metered and delivered dose out of HANDIHALER® using commercial SPIRIVA® capsules purchased from our local pharmacy. From the blister holding 5 capsules one capsule was withdrawn and the remaining 4 capsules were put 4 days into 40° C. and 75% Rh. The blister containing the 4 capsules was then put in an exicator for 2 h before tests were performed. The test was performed with 4 kPa pressure drop over the HANDIHALER® at room temperature and laboratory ambient conditions.

Test S4
An in-use stability test of the aerodynamic fine particle fraction of metered and delivered dose out of HANDIHALER® using commercial SPIRIVA® capsules purchased from our local pharmacy. From the blister holding 5 capsules one capsule was withdrawn and the remaining 4 capsules were put 13 days into 40° C. and 75% Rh. The blister containing the 4 capsules was then put in an exicator for 2 h before tests were performed. The test was performed with 4 kPa pressure drop over the HANDIHALER® at room temperature and laboratory ambient conditions.

Test S5
An in-use stability test of the aerodynamic fine particle fraction of metered and delivered dose out of HANDIHALER® using commercial SPIRIVA® capsules purchased from our local pharmacy. From the blister holding 5 capsules one capsule was withdrawn and the remaining 4 capsules were put 21 days into 40° C. and 75% Rh. The blister containing the 4 capsules was then put in an exicator for 2 h before tests were performed. The test was performed with 4 kPa pressure drop over the HANDIHALER® at room temperature and laboratory ambient conditions.

### High Barrier Seal Tests

Test HBS1
An in-use stability test of the aerodynamic fine particle fraction of metered and delivered dose out of HANDIHALER® using SPIRIVA® formulation from bulk powder loaded during relative humidity below 10% into containers made to act as a high barrier seal, in this case aluminum foils from Alcan Singen Germany and then sealed to absolute tightness. The aluminum containers were put in an exicator for 2 h before the SPIRIVA® powder formulation was loaded from the aluminum containers into the originator capsules at a relative humidity below 10%. The test was performed with 4 kPa pressure drop over the HANDIHALER® at room temperature and laboratory ambient conditions.

Test HBS2
An in-use stability test of the aerodynamic fine particle fraction of metered and delivered dose out of HANDIHALER® using SPIRIVA® formulation from bulk powder loaded during relative humidity below 10% into containers made to act as a high barrier seal, in this case aluminum foils from Alcan Singen Germany and then sealed to absolute tightness. The sealed aluminum containers were put into climate chambers for 7 days at 40° C. and 75% Rh. The aluminum containers were put in an exicator for 2 h before the SPIRIVA® powder formulation was loaded from the aluminum containers into the originator capsules at a relative humidity below 10%. The test was performed with 4 kPa pressure drop over the HANDIHALER® at room temperature and laboratory ambient conditions.

Test HBS3

An in-use stability test of the aerodynamic fine particle fraction of metered and delivered dose out of HANDIHALER® using SPIRIVA® formulation from bulk powder loaded during relative humidity below 10% into containers made to act as a high barrier seal, in this case aluminum foils from Alcan Singen Germany and then sealed to absolute tightness. The sealed aluminum containers were put into climate chambers for 14 days at 40° C. and 75% Rh. The aluminum containers were then put in an exicator for 2 h before the SPIRIVA® powder formulation was loaded from the aluminum containers into the originator capsules at a relative humidity below 10%. The test was performed with 4 kPa pressure drop over the HANDIHALER® at room temperature and laboratory ambient conditions.

### C-Haler DPI Tests

A test was also made outside the stability test program to evaluate our proprietary inhaler, the so-called C-haler, in comparison with the HANDIHALER® using a tiotropium formulation. The C-haler cartridge used high barrier seals made out of aluminum foils from Alcan Singen Germany and the containers where filled volumetrically with 5 mg of the SPIRIVA® powder formulation in bulk. The test was performed using a 4 kPa pressure drop over the C-haler at room temperature and laboratory ambient conditions. The results from the Andersen impactor tests were calculated on fine particle fraction based on delivered dose as well as on metered dose and converted to FPD. The results are given in Table 1 below.

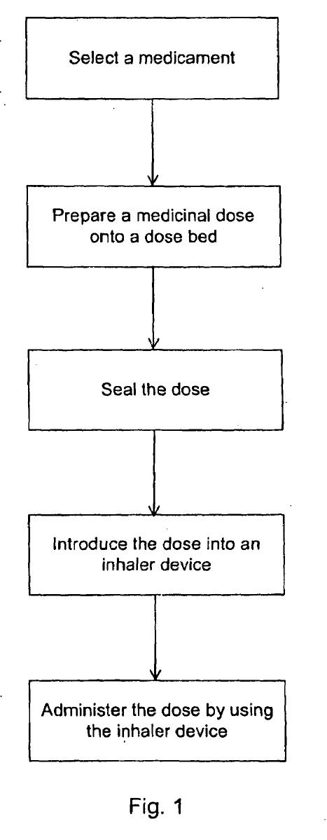

The results of tests S1-5 and HBS1-3 are plotted in FIG. 1. The Y-axis is designated ‘% of commercial SPIRIVA® FPD’. This relates to the FPD out from the HANDIHALER®, where 100% is the FPD from a fresh sample from the pharmacy.

TABLE 1
Inhaled fine particle dose (FPD) <5 μm in %

| Calculation | SPIRIVA ® in HANDIHALER ®, | SPIRIVA ® in |
|---|---|---|
| based on | commercial sample, FPD | C-haler, FPD |
| Metered dose | 18% | 47% |
| Delivered dose | 36% | 56% |

### Conclusion of the Tests Performed on SPIRIVA®

Surprisingly we have found and concluded in our tests that tiotropium is extremely sensitive to moisture and that a conventional packaging into gelatin capsules used for a majority of respiratory products will seriously affect the FPD. The results show that there is a need for a dry, moisture-tight high barrier seal enclosing the tiotropium formulation to preserve the original fine particle fraction. Not so surprisingly in the light of these findings, we have also found that the tiotropium formulation must be properly protected also during the in-use time if further reduction of the FPD shall be avoided. Eliminating the gelatin capsule has an unexpected, big, positive effect on the performance of the SPIRIVA® formulation.

The tests carried out show that the moisture content of the gelatin capsule reduces the FPD out of the HANDIHALER® with approximately 50% from the time of loading the dose into a capsule until the point in time when the product reaches the market. Loading SPIRIVA® doses into dry containers made of materials presenting high barrier seal properties and then storing the loaded containers in 40° C. and 75% Rh, before transferring the SPIRIVA® doses to originator capsules and performing the same tests using HANDIHALER® as before, no change can be detected in the fine particle dose (FPD), even after long periods of time. The FPD of SPIRIVA® in gelatin capsules, however, is further diminishing during the in-use time of the product and the FPD has been shown to drop up to another 20% after 5 days of storage in 40° C. and 75% Rh in an in-use stability test, due to the breaking of the moisture barrier in the opened blister secondary package. Table 1 shows that our propertiary C-haler using high barrier containers shows a 2.6 times higher performance than HANDIHALER® with respect to FPD based on metered dose.

### State of the Art

Metered doses of the SPIRIVA® powder formulation are today at the originator manufacturing site loaded into gelatin capsules. A gelatin capsule contains typically 13-14% water by weight in the dose forming stage and after the capsules have been loaded they are dried in a special process in order to minimize water content. A number of dried capsules are then put in a common blister package. Details about suitable state-of-the-art capsule materials and manufacturing processes may be studied in the German Patent Application DE 101 26 924 A1. The remaining small quantity of water in the capsule material after drying is thus enclosed in the blister package and some water will be released into the enclosed air, raising the relative humidity in the air. The equilibrium between the captured air inside the package and the gelatin capsule will generate a relative humidity inside the blister package that will negatively affect the FPD of tiotropium powder out of the dry powder inhaler.

It is interesting to note that the big majority of dry powder formulations of many kinds of medicaments are not seriously affected by enclosed moisture in the capsule material or by normal storage variations in the relative humidity of the surrounding air. Surprisingly, our investigation has shown tiotropium to be very much different. Tiotropium powder is very much affected by very small amounts of water such that it tends to stick to wall surfaces and to agglomerate. By some mechanisms the FPD becomes less over time. Since the capsules are only used as convenient, mechanical carriers of SPIRIVA® doses, a solution to the moisture problem would be not to use capsules at all, but rather to directly load doses into containers made of dry packaging material with high barrier seal properties during dry ambient conditions, preferably below 10% Rh.

The present invention discloses a dry, moisture-tight, directly loaded and sealed container enclosing a metered dose of tiotropium powder or a pharmaceutically acceptable salt, enantiomer, racemate, hydrate, or solvate, including mixtures thereof, and particularly tiotropium bromide, optionally further including excipients. The term “tiotropium” is in this document a generic term for all active forms thereof, including pharmaceutically acceptable salts, enantiomers, racemates, hydrates, solvates or mixtures thereof and may further include excipients for whatever purpose. The container uses dry, high barrier seals impervious to moisture and other foreign matters and is adapted for insertion into a dry powder inhaler device or the container may be adapted to be a part of an inhaler device.

“Dry” means that the walls of the container are constructed from selected materials such that the walls, especially the inside wall of the container, cannot release water that may affect the tiotropium powder in the dose such that the FPD is reduced. As a logical consequence container construction and materials should not be selected among those suggested in the German publication DE 101 26 924 A 1.

“High barrier seal” means a dry packaging construction or material or combinations of materials. A high barrier seal is characterized in that it represents a high barrier against moisture and that the seal itself is ‘dry’, i.e. it cannot give off measurable amounts of water to the load of powder. A high barrier seal may for instance be made up of one or more layers of materials, i.e. technical polymers, aluminum or other metals, glass, siliconoxides etc that together constitutes the high barrier seal.

A “high barrier container” is a mechanical construction made to harbour and enclose a dose of e.g. tiotropium. The high barrier container is built using high barrier seals constituting the walls of the container.

“Directly loaded” means that the metered dose of tiotropium is loaded directly into the high barrier container, i.e. without first loading the dose into e.g. a gelatin capsule, and then enclosing one or more of the primary containers (capsules) in a secondary package made of a high barrier seal material.

The high barrier containers to be loaded with tiotropium should preferably be made out of aluminum foils approved to be in direct contact with pharmaceutical products. Aluminum foils that work properly in these aspects generally consist of technical polymers laminated with aluminum foil to give the foil the correct mechanical properties to avoid cracking of the aluminum during forming. Sealing of the formed containers is normally done by using a thinner cover foil of pure aluminum or laminated aluminum and polymer. The container and cover foils are then sealed together using at least one of several possible methods, for instance:
using a heat sealing lacquer, through pressure and heat;
using heat and pressure to fuse the materials together;
ultrasonic welding of the materials in contact.

Tiotropium in pure form is a very potent drug and it is therefore normally diluted before dose forming by mixing with physiologically acceptable excipients, e.g. lactose, in selected ratio(s) in order to fit a preferred method of dose forming or loading. Details about inhalation powders containing tiotropium in mixtures with excipients, methods of powder manufacture, use of powder and capsules for powder may be studied in the international publication WO 02/30389 A1.

In a further aspect of the invention tiotropium may be mixed or formulated with one or more other pharmacologically active ingredient(s) with an object of combining tiotropium with other medicament(s) to be used in a treatment of respiratory disorders. The present invention encompasses such use of tiotropium when a combination of tiotropium and other medicaments are deposited and sealed into a dry, moisture-tight high barrier container intended for insertion into a DPI for inhalation by the user. Examples of interesting combinations of substances together with tiotropium could be inhalable steroids, nicotinamide derivatives, beta-agonists, beta-mimetics, anti-histamines, adenosine A2A receptors, PDE4 inhibitors, dopamine D2 receptor agonists.

The sealed, dry, high barrier container of the invention that is directly loaded with a formulation of tiotropium may be in the form of a blister and it may e.g. comprise a flat dose bed or a formed cavity in aluminum foil or a molded cavity in a polymer material, using a high barrier seal foil against ingress of moisture, e.g. of aluminum or a combination of aluminum and polymer materials. The sealed, dry, high barrier container may form a part of an inhaler device or it may be a separate item intended for insertion into an inhaler device for administration of doses.

An inhaler providing a prolonged delivery of a dose during the course of a single inhalation constitutes a preferred embodiment of an inhaler for the delivery of the tiotropium powder formulation, e.g. SPIRIVA®. An Air-razor method as described in our publication US 2003/0192539 A1 is preferably applied in the inhaler to efficiently and gradually aerosolize the dose when delivered to the user. Surprisingly enough, applying an inhaler for a prolonged delivery and using the Air-razor method on a dose comprising tiotropium in SPIRIVA® formulation results in a FPD at least twice as big as that from the state-of-the-art HANDIHALER®.

1. medical product comprising a single dry powder dose of tiotropium directly loaded into a container adapted for administration by a dry powder inhaler, wherein
the medical product comprises a moisture-tight seal foil fixed to a dry, moisture-tight container;
the dry, moisture-tight container and the moisture-tight seal foil together form a dry, moisture-tight, barrier seal;
the moisture-tight barrier seal prevents ingress of moisture into the single powder dose whereby the original fine particle fraction of the single powder dose is preserved for at least seven days;
the medical product is designed for enabling gradual aerosolization of the powder dose during delivery; and
a delivered fine particle dose of tiotropium from said medical product is generally independent of variations in ambient humidity conditions.

2. The medical product according to claim 1, wherein
the original fine particle fraction of the single powder dose is preserved for at least fourteen days.

3. The medical product according to claim 1, wherein the container itself does not emit water.

4. The medical product according to claim 1, wherein the delivered fine particle dose of tiotropium constitutes at least 56% of a delivered dose.

5. The medical product according to claim 1, wherein the delivered fine particle dose of tiotropium constitutes at least 47% of a metered dose.

6. The medical product according to claim 1, wherein the tiotropium substance consists of one or more physiologically acceptable salts of tiotropium.

7. The medical product according to claim 1, further comprising lactose as an included excipient.

8. The medical product according to claim 1, wherein the dry, moisture-tight seal foil is constituted of formed or flat aluminum foils, optionally laminated with polymers.

9. The medical product according to claim 1, wherein the dry, moisture-tight container is constituted of a cavity molded from a polymer material.

10. The medical product according to claim 1, wherein the medical product is a separate part adapted for insertion into a dry powder inhaler

11. The medical product according to claim 1, wherein the medical product is intended for use in the treatment of respiratory disorders.

12. A medical product comprising tiotropium and at least one additional active pharmaceutical ingredient and optionally including at least one excipient in a single dry powder medical combination dose directly loaded into a container, wherein the medical product comprises a moisture-tight seal foil fixed to a dry, moisture-tight container;
the dry, moisture-tight container and the moisture-tight seal foil together form a dry, moisture-tight barrier seal;
the moisture-tight barrier seal prevents ingress of moisture whereby the original fine particle fraction of the single combination dose is preserved for at least seven days;
the combination dose is adapted for administration by a dry powder inhaler;
the medical product is designed for enabling gradual aerosolization of the powder dose during delivery;
a delivered fine particle dose of the single combination dose is generally independent of variations in ambient humidity conditions, and
the at least one additional active pharmaceutical ingredient is selected from the following groups of substances: inhalable steroids, nicotinamide derivatives, beta-agonists, beta-mimetics, anti-histamines, adenosine A2A receptors, PDE4 inhibitors, dopamine D2 receptor agonists.

13. The medical product according to claim 12, wherein the original fine particle fraction of the single combination dose is preserved for at least fourteen days.

14. The medical product according to claim 12, wherein the container itself does not emit water.

15. The medical product according to claim 12, wherein the delivered fine particle dose of the single combination dose at least 56% of a delivered dose.

16. The medical product according to claim 12, wherein the delivered fine particle dose of the single combination dose constitutes at least 47% of a metered dose.

17. The medical product according to claim 12, wherein the tiotropium substance consists of one or more physiologically acceptable salts of tiotropium.

18. The medical product according to claims 12, wherein an included excipient is lactose.

19. The medical product according to claim 12, wherein the dry, moisture-tight seal foil is constituted of formed or flat aluminum foils, optionally laminated with polymers.

20. The medical product according to claim 12, wherein the dry, moisture-tight container is constituted of a cavity molded from a polymer material.

21. The medical product according to claim 12, wherein the medical product is a separate part adapted for insertion into the dry powder inhaler.

22. The medical product according to claim 12, wherein the medical product is intended for use in the treatment of respiratory disorders.
